(19) United States
(12) Patent Application Publication
(10) Pub. No.: US 2005/0124911 A1
(43) Pub. Date: Jun. 9, 2005

(54) Means and method for treating dizziness and balance disturbances

(75) Inventors: Klaus Ehrenberger, Wien (AT); Stefan Thurner, Wien (AT)

Correspondence Address: NIXON & VANDERHYE, PC, 1100 N GLEBE ROAD, 8TH FLOOR, ARLINGTON, VA, 22201-4714, US

(73) Assignee: Weluga-Pharm Anstalt, Nendeln (LI)

(21) Appl. No.: 11/002,955

(22) Filed: Dec. 3, 2004

(30) Foreign Application Priority Data
Feb. 3, 2004 (CH) 00158/04
Dec. 6, 2003 (CH) 02082/03

(51) Int. Cl. A61B 13/00

(52) U.S. Cl. 600/558

(57) ABSTRACT

The invention relates to a means in the form of a piece of information which can be reproduced on suitable reproduction equipment and can be visually registered, which information exists as a sequence of images, for example as a video sequence, and, when reproduced on a display, is non-linearly displaced at such a rate that the information can still just be registered by the observer's eye.

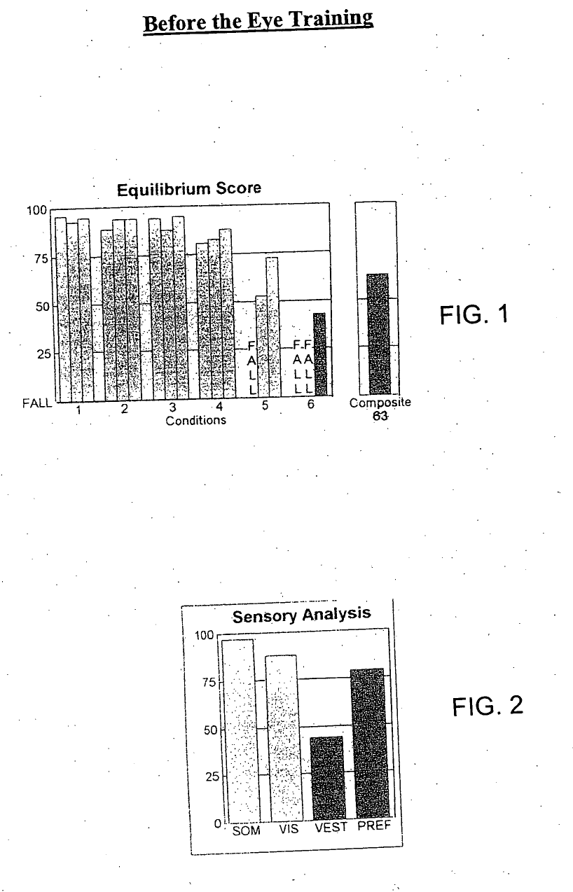

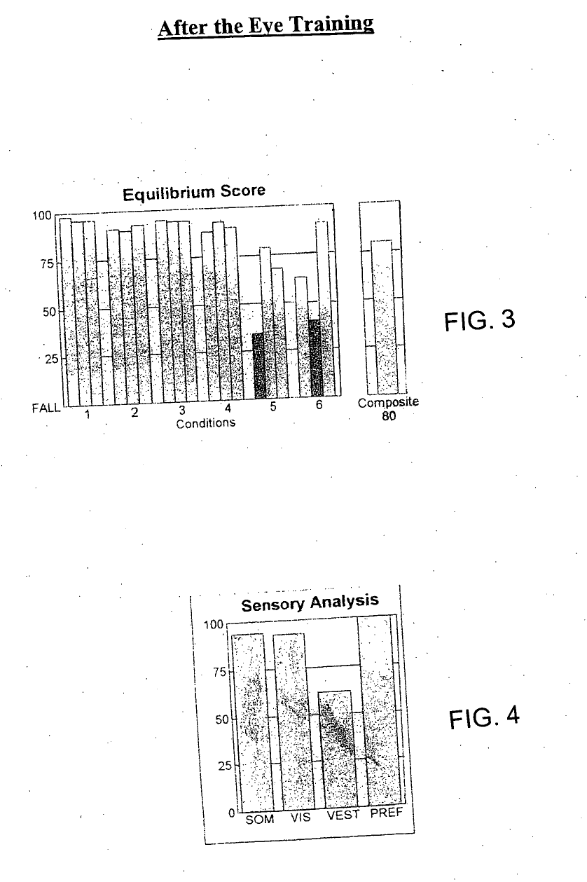

### FIELD OF THE INVENTION

The invention relates to a means and method for treating dizziness and balance disturbances.

### INTRODUCTION

65% of people over 60 years of age complain of dizziness or poor balance and troubles associated therewith. In Austria the proportion of people over 60 is 21% (1.7 million people). This corresponds to about 1.1 million people who are affected by dizziness or poor balance. Age-related dizziness complaints are a manifestation of a gradually decreasing functionality of the orientation system. The intact biological function of spatial orientation is characterised by a complex interaction between numerous control mechanisms which register the space (primarily balance organs, eyes, somato sensory system) and which purposefully control the musculature of the motor control system so an upright posture and movement in space counter to gravity and other acceleration forces is possible. Complex functions guarantee a rapid reaction in numerous degrees of freedom and this makes it possible for the individual to react quickly and purposefully to unexpected environmental influences. Speed and dynamics of complex functions generally decrease with age. The aging phenomenon also affects the orientation system with the above-described, clinically manifest, functional effects. Consistent training counteracts the consequences of ageing processes on complex biological systems (for example cardiovascular training, swallow-reflex training, etc). This also applies to the orientation system. Exercise for the elderly and apparatus-assisted stability-promoting movement exercises primarily use the somatosensory control input for targeted system training. However, these training concepts are opposed by age-related joint problems, muscular atrophy and in many case lack of motivation, so fall-induced injuries among older people increase as a function of the increasing age of society.

U.S. Pat. No. 6,540,355 relates to a method, a device and associated software for exercising the human eye with a monitor, on which a plurality of objects are projected in such a way that portions of the objects have a contrast which changes at a rate of two or fewer oscillations per second. The objects preferably consist of triangles, rectangles or lines with contrasting colours, for example black/white, red/green, etc., arranged in pairs. Preferably, a large number of symbols consisting of a plurality of lines arranged parallel and side by side is statically projected onto the monitor. The lines of the symbols are—with the exception of one line—of the same length. The vision efficiency may then be calculated from the number of correctly recognised symbols. The described method is used to test the power of vision of an individual directly at the computer workstation. This allows, for example, the adjustments of the monitor to be adapted to the user. The exercises are also used to reduce the fatigue of the eyes. A characteristic of the method of U.S. Pat. No. 6,540,355 is that the projected images are static, i.e. do not move on the monitor.

### OBJECT OF THE INVENTION

The object of the present invention is therefore to provide a means and method for treating dizziness and balance disturbances. A further object is to provide a method of manufacturing a means for treating dizziness and balance disturbances. A still further object is to provide a method of producing a means for treating dizziness and balance disturbances.

### DESCRIPTION

According to the invention the object is achieved in that the means is a piece of information which can be reproduced on suitable reproduction equipment and can be visually registered, in that the information, when reproduced on a display, exists as a sequence of images, for example as a video sequence, in which the information is stochastically displaced in any desired directions at such a rate that the information can still just be registered by the observer's eye. Advantageous configurations of the method are defined in the sub-claims. The means has the advantage that a movement structure is imposed on the observer, for example an older person in whom the movement structure has slowed down, as exists in healthy people. This is a completely new method for improving the balance capacity and of treating balance disturbances or dizziness, which frequently occur in older people, in particular. The means according to the invention is therefore a visual therapy for influencing physiological processes such as the orientation system by using the complex system.

Basically, the information existing as a sequence of images or stored information can be a sequence of images comprising a plurality of letters or words or different objects. This is of importance as it has to be ensured that the eyes of the patient follow the moving text or the moving objects. The tests carried out by the inventors have surprisingly shown that a single image, which is moved on a display, is not capable of training the movement pattern of the eye. It is not sufficient for the information to be stochastically moved to and fro and up and down on the display, rather it must be ensured that the observer follows the jerkily displaced information with his eyes. This may best be achieved in that the information is a text of a specific length, preferably a text comprising a plurality of lines. If the information exists as a readable text then this has the advantage that the reader is encouraged to follow information jumping around on the display if he wishes to register the text. At the same time a check is easily possible if the reader has to read the text out loud. Basically it is conceivable to simultaneously record the text when a microphone is provided. The recorded text allows the doctor to check whether the eye training has been carried out properly. It is also possible to carry out the training to improve the balance capacity at home.

The position of the text on the display is advantageously stochastically varied at such a rate that the text can still just be read by the observer. In practice the situation is such that the text appears at a first location during a first time period, at a second location, which is spaced apart from the first location, during a second subsequent time period, at a third location, which is spaced apart from the second location, during a third subsequent time period and so on, the direction of displacement between a location n (of period n) and a location n+1 (of period n+1) stochastically varying. In the process the displacement distance is in each case preferably also varied within a predetermined range.

The text is expediently varied at such a rate that it is only stationary for fractions of a second in each case. However, the standstill times should be sufficient for the observer to be able to perceive the information with the senses.

The time period of the standstill is in each case preferably between 10 and 2,000, preferably between 80 and 1,000 and most preferably between 80 and 800 milliseconds.

The means according to the invention can exist as a video sequence in a format that can be reproduced on a computer (*.mpeg, *.avi, *.wmf, etc.). This has the advantage that the video sequence can be electronically transmitted via the Internet. However, the means according to the invention can be stored on a data carrier (disk, CD-ROM, magnetic tape). It is also conceivable, however, that only one program is available which stochastically varies the position on the display of any desired text which can be determined by the user in the manner according to the invention.

According to a further aspect of the invention, a method is claimed for producing a means according to any one of claims 1 to 12, which is characterised in that a piece of information which can be depicted on a display, preferably a text, is stochastically displaced on the display, using a program, at such a rate that the information (text) can still be registered or read by the observer's eye. This program can basically be made available to the user so he can select any desired text with which he wishes to carry out the eye training. The program can also be used to displace the information in any desired directions in a time sequence and at random displacement distances within a certain range and to record the generated pattern on a data carrier, for example a video cassette, DVD or the like, as a sequence of images (video sequence).

In a therapeutic method for treating dizziness and/or balance disturbances and/or for improving the balance capacity in older people, a piece of information which can be reproduced on suitable reproduction equipment and can be visually registered and registered by the other senses, is stochastically displaced during reproduction on a display at such a rate that the information is stationary only for fractions of a second in each case. In the process, it must, however, still be possible for the observer's eye to register or read the information. According to a particularly preferred variation of the method, a text comprising a plurality of largely different words is stochastically moved on a display. In this case the patient has to attempt to read the text out loud. The eye training can be checked by the patient himself or by a third party by the process of reading out loud. It is also conceivable to record the voice of the patient so a subsequent check or a remote check is possible. The text displayed can be a one line or a multi-line text.

Training of the Orientation System Via the Visual Check Input:

Eye movements are a good reflection of the complex structure of the orientation system with its numerous degrees of freedom. The dynamics of the system corresponds to a non-linear system with a “self-similar” or “fractal” structure, of which the inherent functional order can be well depicted using the method of fractal geometry. This functional structure changes with age with alarming contraction of necessary degrees of freedom. According to the invention, it is accordingly proposed to re-program the “fractal” structure in the orientation system using non-linear eye movements and to thus return a “youthful” function to the system via eye training. The visual stimulation takes place via a text-image with a non-linear (stochastic) movement pattern which is projected onto a screen. The patient has to read the text out loud to ensure that the eyes are following the movement pattern. This training method is also reasonable for wearers of glasses and is completely independent of the condition of the movement apparatus of the body. It was possible to prove the high clinical value of this visual dizziness therapy in a “Proof of concept” study. The innovative core to this therapy is the movement pattern which is characterised as follows:

The Movement Pattern

It was found from the movement patterns of the body movements when standing that the “degree of self-similarity” (fractality) decreases with age in these patterns. It is therefore proposed to impose “self-similar” patterns on the balance system again. The movement pattern is characterised as follows:

The image/text moves in two dimensions. The movement of the centre of the image is described by x(t) and y(t) components, where t is the time. After its appearance, the position of the image/text remains constant for a period of Δt seconds (Δt is between 10 and 2,000, preferably between 80 and 1,000, and most preferably between 80 and 800 milliseconds). After this stationary period the position of the image is displaced to a new position. The displacement in the x direction is δₓ and δ_{y} in the y direction. The image remains at this position for Δt seconds again, is subsequently displaced again, etc.

The movement pattern is characterised by the spectrum S(ω) of the movement components x and y (absolute square of the Fourier transform), where ω is the frequency. The spectrum follows (over at least half an order of magnitude of the frequency) a power law, i.e. S=cω^{-β}. Here c is any desired constant and β a real number between 0 and 4. This connection also applies to the increments of x and y, i.e. δₓ(t)=x(t)−x(t−Δt) and δ_{y}(t)=y(t)−y(t−Δt), (of course with different c and β). The term t-Δt represents the instant of the Δt seconds before t is or was, which results in absolute values from the specially selected time resolution or update rate of the system.

The movement pattern is also characterised by the function F(τ)={[x(t+τ)−x(t)]²}ₜ=dτ^{2H}, wherein d is any constant and H is the Hurst exponent which is between 0 and 1. {.}ₜ represents the average over time. This law should exist at least over half an order of magnitude in τ. The same applies to component y.

Amplitudes (mean values and standard deviation of δₓ and δ_{y}) and rate (update rate or time increment Δt) are variable and individually adjusted.

In the literature δₓ and δ_{y} are sometimes called “correlated noise” or “anti-correlated noise” or “fractal noise” or “fractional Brownian noise”.

### EMBODIMENT

The therapy used in a study consisted of 10 half-hour sessions. The tests showed that a daily half-hour exercise drastically intensifies the improvement in patients suffering from dizziness and training effect in people with poor balance.

### DESCRIPTION OF THE ENCLOSURES

The accompanying graphics relate to the quantative measurements of the balance capacity in patients suffering from dizziness before and after treatment.

FIG. A shows the rate of the balance capacity during six different tests which were each repeated three times. A balance factor (composite) is calculated from these measurements. This factor is a measure of the balance capacity. In healthy young people this quantity is greater than 70. Over the course of treatment this quantity could be drastically improved in patients (from approx. 60 to approx. 80).

FIG. B shows the functionality of individual sensory inputs. SOM is the somatosensory input, VIS the visual input and VEST the vestibular input. The value is given as between 0 and 100 and shown in colour: poor=red, grey=normal case, green=good. In both patients the vestibular input before treatment is poor and can only be dramatically improved by visual training, however.

The invention relates to a means in the form of a piece of information like a text which can be reproduced on suitable reproduction equipment and can be visually registered, which information exists as a text, for example in the form oa a video sequence, which, when reproduced on a display, shows the text being stochastically displaced at such a rate that the information can still just be registered by the observer's eye but stands still only for fractions of a second. The time period of the standstill is in each case advantageously between 8 and 150, preferably between 80 and 200, and most preferably between 80 and 150 milliseconds.

1. Means, in particular for treating dizziness and/or balance disturbances and/or for improving the balance capacity in older people,
characterised in that the means is a piece of information that can be reproduced on a display by suitable reproduction equipment, which information is stochastically displaced on the display at such a rate that the information can still be registered by the observer's eye.

2. Means according to claim 1, characterised in that, the information, when reproduced on a display, is displaced stochastically at such a rate that the information can still be registered by the observer's eye.

3. Means according to claim 1, characterised in that the information is a text of a specific length, in particular a text with a plurality of lines.

4. Means according to claim 3, characterised in that the position of the text on the display is varied in any desired direction at such a rate that the text can still just be read by the observer.

5. Means according to claim 1, characterised in that the information on the display appears at a first location during a first time period, at a second location, which is spaced apart from the first location, during a second subsequent time period, at a third location, which is spaced apart from the second location, during a third subsequent time period and so on, the direction of displacement between a location n (of period n) and a location n+1 (of period n+1) randomly varying.

6. Means according to claim 5, characterised in that the displacement distance is varied within a predetermined range.

7. Means according to claim 1, characterised in that the information is varied at such a rate that the information is in each case stationary for only fractions of a second up to a maximum of two seconds.

8. Means according to claim 7, characterised in that the time period of the standstill is in each case between 10 and 2,000, preferably between 80 and 1,000, and most preferably between 80 and 800 milliseconds.

9. Means according to claim 1, characterised in that the movement pattern of the information is characterised by the spectrum S(ω) of the movement components x and y (absolute square of the Fourier transform), where ω is the frequency and S(ω) follows a power law of the type S=cω^{-β}, wherein c is any constant and β is a real number between 0 and 4.

10. Means according to claim 9, characterised in that the movement pattern is further characterised by the function F(τ)={[x(t+τ)−x(t)]²}ₜ=dτ^{2H}, where d is any desired constant and H is the Hurst exponent which is between 0 and 1.

11. Means according to claim 1, characterised in that the means is present in the form of a data carrier with said piece of information, which is stored on the data carrier, can be reproduced on suitable reproduction equipment and can be visually registered.

12. Data carrier on which the means according to claim 1 is stored.

13. Method for producing a means in the form of a data carrier according to claim 1, characterised in that a piece of information which can be depicted on a display, for example a text, is stochastically displaced on the display, using a program, at such a rate that the information can still be registered by the observer's eye.

14. Method according to claim 13, characterised in that the movement pattern generated by the program is recorded on a further data carrier, for example a video cassette, DVD, memory stick or the like.

15. Method according to claim 13, characterised in that the information is either a text consisting of a plurality of words.

16. Use of a piece of information, which can be visually registered and depicted on a display, which is characterised in that the information, on reproduction on a display, is displaced at such a rate that the information can still just be registered by the observer's eye, for producing a means for treating dizziness and/or balance disturbances and/or for improving the balance capacity in older people.

17. Use of a means according to claim 1 for treating dizziness and/or balance disturbances and/or for improving the balance capacity in older people.

18. Method for treating dizziness and/or balance disturbances and/or for improving the balance capacity in older people, characterised by the following method steps:
a) providing a display
b) projecting a piece of information like a text, which can be visually registered, onto the display and
c) stochastically varying the position of the information on the display at such a rate that the information can still be registered by the observer's eye.

19. Method according to claim 18, characterised in that the information consists of words, and/or of a text of a specific length.

20. Method according to either claim 18, characterised in that the patient has to read the text out loud.

21. Method according to claim 20, characterised in that the spoken text is recorded.
